(19) United States
(12) Patent Application Publication
(10) Pub. No.: US 2019/0046698 A1
(43) Pub. Date: Feb. 14, 2019

(54) WOUND DRESSING ASSEMBLY

(71) Applicant: LOHMANN & RAUSCHER GMBH, SCHONAU AN DER TRIESTING (AT)

(72) Inventor: GUNNAR LOSKE, AHRENSBURG (DE)

(21) Appl. No.: 15/760,872

(22) PCT Filed: Sep. 6, 2016
(86) PCT No.: PCT/EP2016/001504

§ 371 Date: Aug. 2, 2018

(30) Foreign Application Priority Data
Sep. 17, 2015 (DE) 20 2015 006 570.8

(51) Int. Cl. A61M 1/00; A61F 13/02

(52) U.S. Cl. CPC A61M 1/0088; A61F 5/445; A61F 13/0216; A61F 13/0206

(57) ABSTRACT

The invention relates to a wound dressing assembly, in particular for dressing wounds in the region of enteroatmospheric fistulas, comprising an in particular gas-tight covering device for covering the wound and/or a skin region surrounding the wound, which covering device inhibits the penetration of fluids, characterized by an opening in the covering device and a reinforcing device, which is associated with the opening and is connected to the covering device in a fluid-tight manner and counteracts deformation of the covering device in the region of the opening, wherein a vacuum can be produced in a space bounded by the covering device, by the skin or wound covered by the covering device, and by the reinforcing device.

### CROSS-REFERENCE TO RELATED APPLICATIONS

This is a national phase application from PCT Patent Application Ser. No. PCT/EP2016/001504 filed on Sep. 6, 2016, which claims priority to German Patent Application Serial No. 20 2015 006 570.8 filed on Sep. 17, 2015, each of which is incorporated herein by reference in its entirety.

### BACKGROUND OF THE INVENTION

### 1. Field of the Invention

The invention relates to a wound treatment arrangement, in particular for the treatment of wounds in the area of enteroatmospheric fistulae, having a covering device, in particular being fluid-tight, which inhibits the passage of fluids, and covers the wound and a skin area surrounding the wound.

### 2. Description of Related Art

Enteroatmospheric fistulae form a connection between an intestinal lumen and the skin, across which the intestinal contents can be evacuated through a skin opening. The fistulae form as a complication in the course of abdominal surgery. During treatment, the intestinal contents can be drained off across the fistula into a pouch. At the same time, the need arises to treat wounds formed around the fistula opening.

### BRIEF SUMMARY OF THE INVENTION

The invention has as its objective providing wound treatment arrangements, by means which, on the one hand, an effective treatment of wounds that exist in the area of fistulae openings and, on the other hand, a desired treatment of the patient, while taking advantage of the fistula opening, can be assured.

According to the invention, this objective is achieved by an enhancement of wound treatment arrangements of the type stated in the preamble, which is substantially characterized in that the covering device comprises an opening, which is associated with a reinforcing device that is connected thereto and counteracts deformation of the covering device in the area of the opening, allowing the generation of a negative pressure in a space delimited by the covering device, the skin or wound covered thereby, and the reinforcing device.

If the wound treatment arrangement according to the invention is designed in such a way that the opening of the covering device is arranged above the fistula opening, the treatment of a wound in the area of the fistula opening can be accomplished using the negative pressure therapy, while simultaneously, using the reinforceing device, assuring that the fistula opening per se remains accessible, so that desired treatments, such as, for instance, drainage of the evacuating intestinal contents into a pouch, can take place through the fistula opening and through the reinforcing device.

In other words, the invention allows improving both drainage of the intestinal contents into a pouch and wound treatment and facilitating care of the patient. In doing so, the invention can also be used for catching wound secretions, urine or the like or for the treatment of open abdominal wounds, for which it is intended to carry out flushing by way of a dressing opening, for instance. It can be combined with so-called vacuum sponge dressings, which are utilized in the negative pressure treatment of wounds and expands their applicability in therapy.

The opening of the covering device may be circular. It may, however, have other shapes, too, for example rectangular or ellipsoidal shape. In any case, in the treatment, the opening should be placeable above the fistula opening so that fistula contents and intestinal contents can be evacuated via this route.

In a preferred embodiment of the invention, the covering device comprises with a unilaterally open-celled film, the open-celled side of which faces the skin and is encircled by an edge which is connectable to the skin in a fluid-tight manner and is connected thereto in a fluid-tight manner.

Therein, unilaterally open-celled film refers to a double-layered or multi-layered film, one boundary surface of which is formed by a continuous fluid-tight film and in which a space formed between individual film layers is in communication with the surrounding area via an opening formed in another film layer. Therein, a film layer provided with cells forms the open-celled side of the covering device. The cells may be designed as so-called three-dimensional cells, which extend funnel-shaped into a space formed between the film layers. If the space formed between the film layers is in communication with a negative pressure generating device via a line connected to a fluid-tight (outer) film layer in a fluid-tight manner, uniform pressure distribution in the space formed between the covering device and the skin or the wound can take place via the unilaterally open-celled film. This uniform pressure distribution promotes wound healing in this area.

The reinforcing device of a wound treatment arrangement according to the invention may comprise a reinforcing ring, in particular a plastic ring, which completely encircles the fistula opening during the treatment state and to which the covering device is connected in a fluid-tight manner. For this purpose, the covering device may, for instance, be glued to a boundary surface of the reinforcing ring, which faces away from the wound. The reinforcing ring may comprise polyvinyl, polyurethane and/or silicone. The scope of the invention provides for the reinforcing ring to be manufactured of solid plastic in its entirety. In this arrangement, the ring may have a rectangular cross-section in an axial sectional plane. Another embodiment of the invention provides for the reinforcing ring to have a flange area that can be placed on the skin and a stub area that extends perpendicular thereto, approximately parallel to the ring axis, the covering device preferably resting on a boundary surface of the flange area facing away from the skin and connectable thereto in a fluid-tight manner. The stub area allows the connection of a receiving device, for example pouch-shaped, in which the intestinal contents and/or wound secretions, urine or the like can be drained.

According to another preferred embodiment of the invention, the stub area has, at its axial end that faces away from the flange area, a connecting flange that extends radially outwards. Facing away from the wound (outer surface of the covering device), the reinforcing ring may instead be manufactured with the covering device as a connecting system for a conventional Anus praeter stoma pouch system with a corresponding adapter. As a result, the invention can simultaneously be used as a stoma pressure plate and a vacuum seal dressing.

Within the scope of the invention, it is furthermore preferred to have the reinforcing ring designed deformable, at least in sections. Hence, the reinforcing ring can adapt to the contour of the wound or of the skin surface in the area of the wound. Hence, by generating a negative pressure in the space delimited by the covering device, the skin covered thereby and the reinforcing device, the skin or wound surface is applied in such a way, that even in the area of the reinforcing device in the form of the deformable reinforcing ring, the space is sealed in a fluid-tight manner by application of the reinforcing ring to the skin.

In the last-described embodiment of the invention, the reinforcing ring may be designed, at least in some areas, as a hollow body, which is delimited by a deformable wall and, as the case may be, may be filled using a fluid, via a valve arrangement. In this embodiment, the reinforcing ring can be designed in tubular form having an inner lumen that can be filled using air or another gas. In this arrangement, the wall of the tubular ring may be thin-walled so as to assure good deformability in this manner. The inner lumen of the tubular ring may be fluid-conductively connected to a channel, across which air, another gas or a fluid can be introduced into the inner lumen and the ring can be inflated like a swim ring. The channel may be equipped with a valve, so that the gas or the fluid cannot escape or can only escape in controlled form. As a result of the swim ring-like design, on the one hand, the ring can be pressure-adapted to the wound overlay, on the other hand, during evacuation, a smaller packing dimension can be achieved.

The ring profile may be rounded, circular, rectangular or triangular. The wound-side contact surface may be rounded or else plane. If it is shaped plane, the contact pressure on the wound surface is thereby intended to be more evenly distributed, so that no pressure damage occurs. Preferably the ring profile ends flush with the covering device. In this arrangement, the ring profile may project over the covering device close to the wound and/or distant from the wound.

The treatment of a wound in the area of a fistula opening can be further enhanced if a flexible and fluid-conducting buffer arrangement, to be arranged between the covering device and the skin or the wound, is provided, which surrounds the opening at least in sections and is connected to the reinforcing device. As a result of this enhancement of the invention, during generation of a negative pressure in the space formed between the reinforcing device, the covering device and the skin or the wound, particularly intimate wound contact of the wound treatment arrangement can be achieved via the buffer arrangement in the area of the reinforcing device or of the opening. As a result, sealing in the area of the reinforcing ring, which rests on the wound close to the fistula and consequently of the wound treatment arrangement, is overall enhanced. Hence, vacuum sealing of the wound surface can be accomplished, during which, on the other hand, evacuation of fistula secretions/intestinal contents can take place via a pouch attached above the opening.

At least in sections, the buffer arrangement may be embodied as an open-celled foam, in particular polyurethane foam. As a result of the cells of the polyurethane foam, which are situated closely adjacent to each other, tighter adhesion to the wound surface than with an open-celled film can be produced so that intimate bonding can be achieved, in particular in the area adjoining the fistula opening.

The buffer arrangement may abut the open-celled side of the unilaterally open-celled film in virtually any form. For this purpose, buffer strips, in particular polyurethane foam strips, can be unilaterally provided with an adhesive surface, so that the strips can be glued to the open-celled side of the unilaterally open-celled film by this adhesive surface. In doing so, the suction properties of the open-celled unit of film and polyurethane foam remain unaffected.

The buffer arrangement may comprise a buffer ring encircling the reinforcing device and at least one, preferably two, three or a plurality of buffer arms extending radially outwardly therefrom, allowing the connection to the negative pressure generating device to be made in the area of an end of one of the buffer arms facing away from the buffer ring.

From the opening of the covering device, which should be situated in the middle of the covering surface, a spider web-like structure of polyurethane foam may originate. In another embodiment of the invention, a reticularly radial arrangement having finger-like extensions may be provided. By such an implementation of the buffer arrangement, some stiffening of the covering device can be achieved, which facilitates the planar spreading and placement of the covering device on the wound.

In a preferred embodiment of the invention, the covering device is designed transparent, at least in sections. Through a transparent section of the covering device, wound healing can be monitored. Furthermore, it is possible to provide for the buffer arrangement to comprise letter- and/or number-like identification elements arranged in a transparent area of the covering device. The identification elements show legibly through the preferably film-like covering device. As a result, wound treatment data, for instance, can be retained in the wound treatment arrangement per se, or the change date for a dressing of the wound treatment arrangement can be noted. The type of identification and labeling of the wound treatment arrangement explained above may also be implemented using not open-celled material, such as textile fabrics or gases. Foams and textile fabrics may be dyed.

The strips of the buffer arrangement usable within the scope of the invention, in particular polyurethane strips, should preferably have a width of 2 mm to 10 mm and have a height of up to 10 mm. The buffer ring, situated directly adjacent to the reinforcing device, or strips encircling it or originating from it should be approximately as high as the ring profile. Hence, if a negative pressure is applied to the open-celled polyurethane foam, this foam collapses and thus causes some suction or pressure of the reinforcing device on the wound surface close to the fistula.

A wound treatment arrangement equipped for the treatment of enteroatmospheric fistulae is designed planar like an adhesive dressing. It preferably has a 5 cm×5 cm up to 50 cm×50 cm surface. Preferably, the opening of the covering device will be centrally located and, particularly preferred, be provided with a reinforcing ring. The reinforcing ring can have a diameter of up to 15 cm. The covering device, which is preferably designed as a unilaterally open-celled film, is connected to the ring in a fluid-tight manner. In this arrangement, the unilaterally open-celled film abuts the wound. The corresponding surface of the unilaterally open-celled film may be provided with additional open-celled material, preferably open-celled polyurethane foam in planar form or in strips or lettering. This side may also lie directly on the intact skin. In the edge area, the covering device can then be provided with an adhesive strip, which is glued onto the healthy skin. This bond in the edge area is intended to seal the dressing in a fluid-tight manner towards the edge.

The wound treatment arrangement according to the invention is particularly advantageous for a negative pressure treatment in the area of enteroatmospheric fistulae. In this process, varied open-celled materials (unilateral film/foam) can be combined in combination with a reinforcing device, in particular a reinforcing ring to be placed over a fistula opening. It involves a unilaterally open-celled dressing having a central opening, by means of which, in the area of a wound with a fistula, a negative pressure can be generated using a vacuum-generating system, preferably an electronic pump. As needed, the opening of the wound treatment arrangement can be designed for adaptation to Anus praeter stoma pouches. Expediently, the wound treatment arrangement will be mostly transparent, so that wound evaluation can preferably take place with a wound treatment arrangement lieing thereon. The wound treatment arrangement can be glued directly onto the skin. A particular advantage is the time savings and significant simplification of the complex wound treatment of such wounds.

In all the embodiments of the invention, a preferably pouch-shaped receiving device can be permanently or detachably arranged above the side of the opening facing away from the wound.

The invention will be explained hereinafter with reference to the drawing, to which reference is expressly made regarding all details that are relevant to the invention and that are not specifically emphasized in the description.

### BRIEF DESCRIPTION OF THE DRAWINGS

FIG. 1 depicts a wound treatment arrangement according to a first embodiment of the invention.

FIG. 2a is a sectional view of the wound treatment arrangement according to FIG. 1.

FIG. 2b is the view of claim 2a with a vacuum was applied via tube (5).

FIG. 3a depicts a wound treatment arrangement according to a second embodiment of the invention.

FIG. 3b is a cross-sectional view of the film of FIG. 3a at the level of the letters or numbers.

FIG. 4a depicts an embodiment of a reinforcing device of wound treatment arrangements according to the invention.

FIG. 4b depicts an embodiment of a reinforcing device of wound treatment arrangements according to the invention.

FIG. 4c depicts an embodiment of a reinforcing device of wound treatment arrangements according to the invention.

FIG. 4d depicts an embodiment of a reinforcing device of wound treatment arrangements according to the invention.

### DETAILED DESCRIPTION OF PREFERRED EMBODIMENT

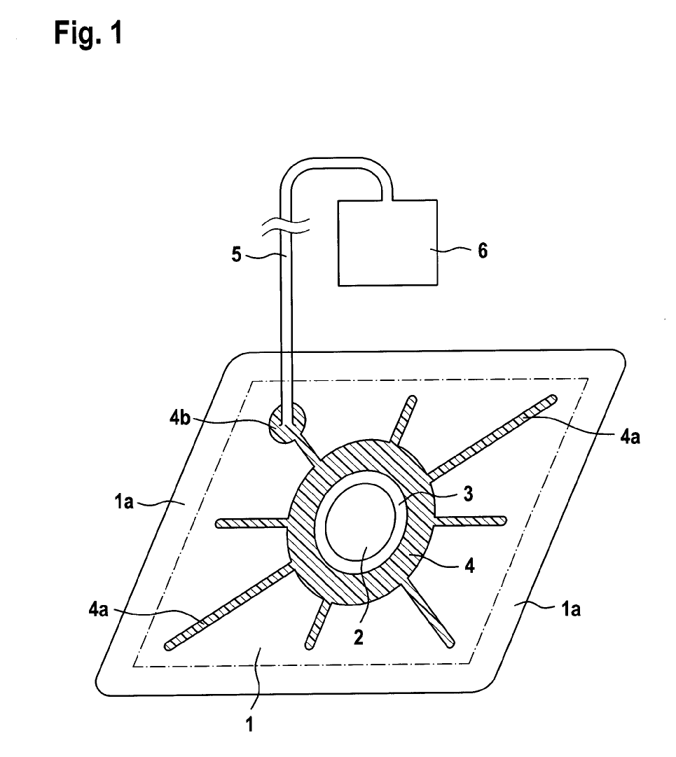

FIG. 1 is a top view of a covering device in the form of a transparent unilaterally open-celled film (1). Peripheral edge (1a) is provided with an adhesive, by means of which the film can be fastened to the skin. In the middle of the film, a circular opening (2) is located. The opening is delimited by a plastic ring (3), which is not fluid-conductively welded to the film. Adjacent to the ring, resting on the open-celled underside, an open-celled polyurethane foam (4) is located, which extends star-like across the film, using finger-like extensions (4a). The open-celled polyurethane foam is fluid-conductively connected to the open-celled side of the film. One of the finger-like extensions of foam (4b) is fluidly-conductively connected to a tube (5), which is connected to a vacuum-generating system (6).

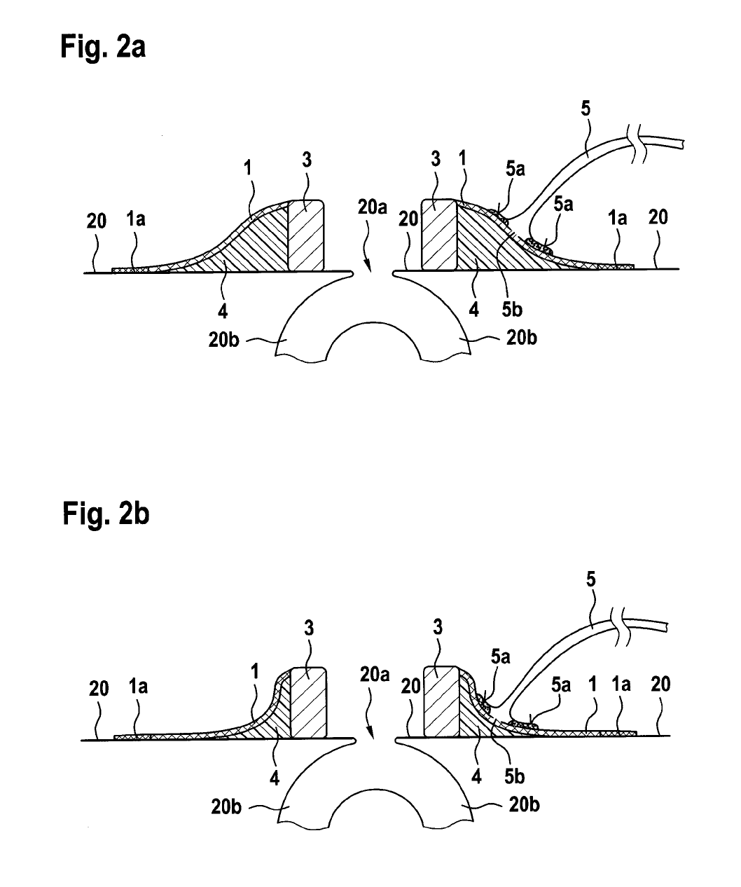

FIG. 2a is a cross-sectional view of FIG. 1 at the level of the ring opening. The open-celled underside of unilaterally open-celled film (1) is deposited on a wound (20), which encloses a fistula opening (20a) to intestine (20b). In the edge area of film (1a), the film is glued down and terminates not fluid-conductively in the wound or skin. Above fistula opening (20a), ring (3) is set on. A fluid-conducting buffer arrangement in the form of an open-celled polyurethane foam abuts the ring. A tube (5) having an adhesive surface (5a) is glued onto the film. Across an opening (5b), the tube is fluid-conductively connected to the open-celled side of the film and to the open-celled foam, so that a vacuum can be applied.

FIG. 2b is the same cross-sectional view as FIG. 2a, with the difference that a vacuum was applied via tube (5) and, as a result thereof, open-celled polyurethane foam (4) has collapsed.

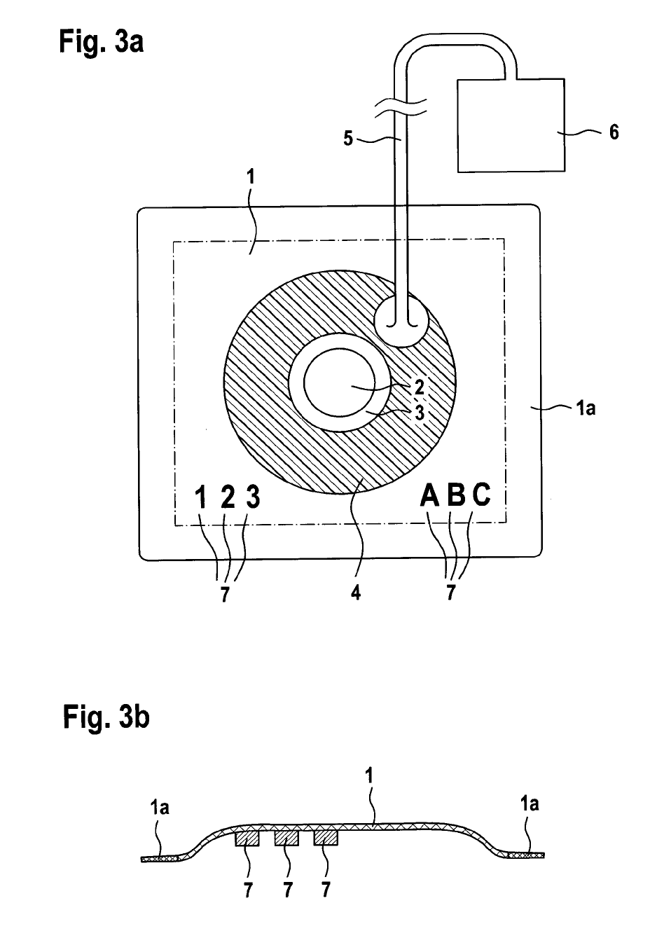

FIG. 3a is a top view of a covering device in the form of a transparent, unilaterally open-celled film (1). Peripheral edge (1a) is provided with an adhesive, by means of which the film can be fastened to the skin. In the middle of the film, a circular opening (2) is located. The opening is delimited by a plastic ring (3), which is not fluid-conductively welded to the film. Abutting the ring, resting on the open-celled underside, a circular open-celled polyurethane foam (4) is located. Foam (4) is fluid-conductively connected to a tube (5), which is connected to a vacuum-generating system (6). On the underside of the open-celled film, letters and numbers (7) made of polyurethane foam strips are applied. Because the film is transparent, they are visible. They are fluid-conductively connected to the film and collapse subject to the exertion of a negative pressure.

FIG. 3b is a cross-section of the film at the level of the letters or numbers, which are fluid-conductively glued to the lower surface in the form of strips of the film.

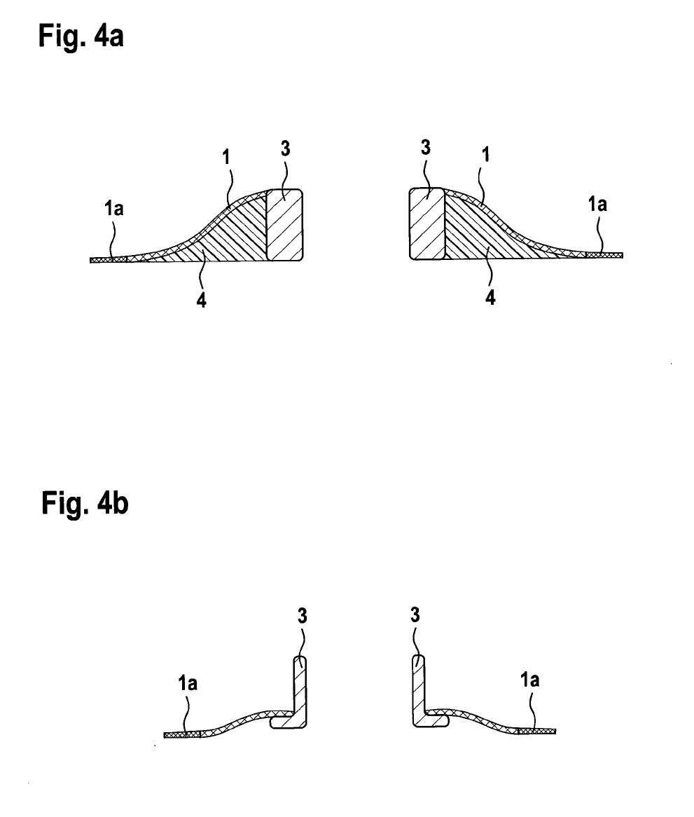

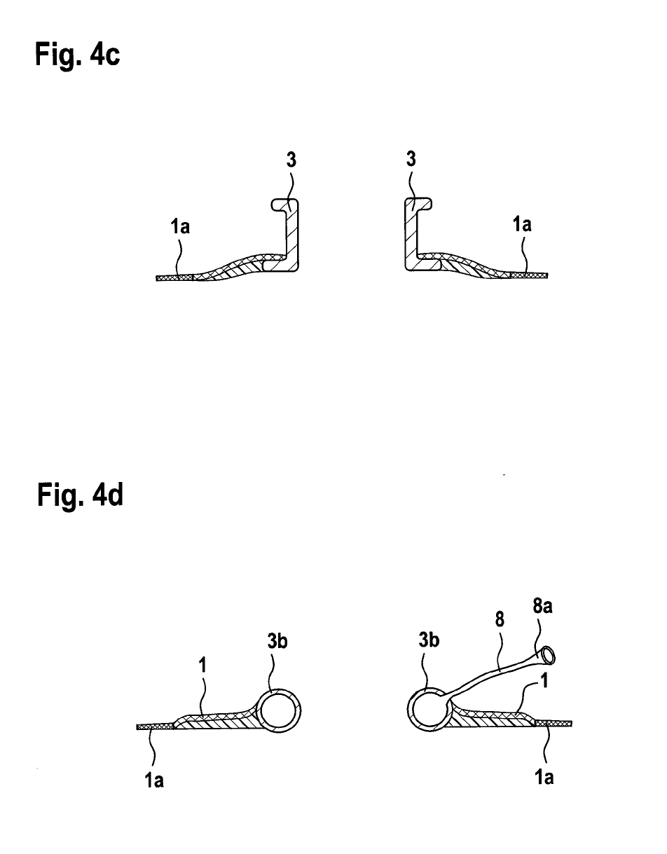

FIGS. 4 a/b/c show different cross-sectional profiles of solidly manufactured plastic ring (3). Unilaterally open-celled film (1) is attached to different locations of the ring, and polyurethane foams (4) of different heights abut the ring. The edge of film (1a) is not fluid-conductive and can be glued to a wound.

In FIG. 4a, ring (3) is a rectangular profile.

In FIG. 4b, ring (3) has an L-shaped profile comprising a narrow edge protruding over film (1). As a result thereof, gluing down of a stoma pouch is to be facilitated.

In FIG. 4c, ring (3) has a clamp-like, C-shaped profile. This profile is designed like the locking profile of stoma pouches so that a pouch could be directly pressed on here.

FIG. 4d is a cross-sectional profile of a plastic ring (3b), which is hollow and can be filled using a gas or a fluid via a channel (8), which is equipped with a valve (8a).

### REFERENCE NUMBER LIST

1 Open-celled Film

1a Peripheral Edge

2 Opening

3 Plastic Ring

4 Polyurethane Foam

4a/4b Finger-like Extensions of the Polyurethane Foam

5 Tube

5a Adhesive Surface

5b Opening

6 Vacuum Generating System

7 Identification Elements

8 Channel

8a Valve

20 Wound

20a Fistula Opening

20b Intestine

1.-13. (canceled)

14. A wound dressing assembly for wounds in the enteroatmospheric fistulae comprising:
a covering device for covering the wound and a skin area surrounding the wound, wherein the covering device inhibits the passage of fluids and comprises an opening; and
a reinforcing device associated with the opening and connected to the covering device in a fluid-tight manner; wherein the reinforcing device counteracts deformation of the covering device in the area surrounding the opening, and wherein the reinforcing device allows for the generation of negative pressure in a space delimited by the covering device, the skin area or the wound, and the reinforcing device.

15. The wound dressing assembly of claim 14, wherein the covering device comprises a unilaterally open-celled film, wherein an open-celled side of the film is configured to face the skin area and is surrounded by an edge that is configured to be connected to the skin area in a fluid-tight manner.

16. The wound dressing assembly of claim 14, wherein the reinforcing device has a reinforcing ring to which the covering device is connected in a fluid-tight manner.

17. The wound dressing assembly of claim 16, wherein the reinforcing ring is a plastic ring.

18. The wound dressing assembly of claim 14, wherein the reinforcing device has a flange area configured to be placed on the skin area and a stub area that extends perpendicular to the flange area, approximately parallel to an axis of the ring-.

19. The wound dressing assembly of claim 18, wherein the stub area has, at an end that faces away from the flange area, a connecting flange that extends radially outwards.

20. The wound dressing assembly of claim 16, wherein the reinforcing ring is deformable.

21. The wound dressing assembly of claim 20, wherein at least a portion of the reinforcing ring is a hollow body delimited by a deformable wall.

22. The wound dressing assembly of claim 21, wherein the deformable wall is configured to be filled with a fluid via a value arrangement.

23. The wound dressing assembly of claim 14, further comprising a flexible and fluid-conducting buffer that surrounds at least a portion of the opening.

24. The wound dressing assembly of claim 23, wherein the flexible and fluid-conducting buffer is connected to the reinforcing device.

25. The wound dressing assembly of claim 23, wherein the flexible and fluid-conducting buffer comprises an open-celled foam.

26. The wound dressing assembly of claim 25, wherein the open-celled foam is polyurethane foam.

27. The wound dressing assembly of claim 23, wherein the flexible and fluid-conducting buffer comprises a buffer ring encircling the reinforcing device and at least one buffer arm extending radially outward from the buffer ring.

28. The wound dressing assembly of claim 23, wherein at least a portion of the covering device is transparent and the buffer comprises identification elements arranged in the transparent portion of the covering device.

29. The wound dressing assembly of claim 14, further comprising a device for generating negative pressure in fluid communication with the connecting device.

30. The wound dressing assembly of claim 29, wherein the device for generating negative pressure is connected to the covering device via a connecting device.

31. The wound dressing assembly of claim 14, further comprising a receiving device attached to the reinforcing device.

32. The wound dressing assembly of claim 31, wherein the receiving device is pouch-shaped.

33. The wound dressing assembly of claim 31, wherein the receiving device is detachably attached to the reinforcing device.

34. The wound dressing assembly of claim 14, wherein the covering device is gas-tight.
